(19) United States
(12) Patent Application Publication
(10) Pub. No.: US 2005/0176073 A1
(43) Pub. Date: Aug. 11, 2005

(54) Cell proliferation associated with CCX CKR expression

(75) Inventors: Yu Wang, Redwood City, CA (US); Brett Premack, San Francisco, CA (US); Thomas Schall, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ChemoCentryx, Inc., San Carlos, CA (US)

(21) Appl. No.: 10/773,380

(22) Filed: Feb. 6, 2004

(51) Int. Cl. G01N 33/574

(52) U.S. Cl. 435/7.23

(57) ABSTRACT

Methods for screening agents to identify an agent that modulates CCX CKR proliferative activity are provided. Tissue generation methods are also disclosed that involve activating CCX CKR expression and activity levels, as are methods for promoting angiogenesis. Methods for treating various diseases associated with cell proliferation are also described.

### BACKGROUND

Chemokines are a class of cytokines that have been identified as playing a major role in a large number of distinct and diverse biological processes. For instance, many chemokines have the ability to cause chemotactic migration of distinct cell types, including monocytes, neutrophils, T lymphocytes, basophils and fibroblasts. Some chemokines are involved in inflammatory responses. Examples of the types of proinflammatory activity that certain chemokines are involved in include: stimulation of histamine release, lysosomal enzyme and leukotriene release, increased adherence of target immune cells to endothelial cells, enhanced binding of complement proteins, induced expression of granulocyte adhesion molecules and complement receptors, and respiratory burst. Certain other chemokines have been found to inhibit hematopoietic stem cell proliferation, and to inhibit endothelial cell growth and proliferating keratinocytes.

Known chemokines are typically assigned to one of four subfamilies based on the arrangement of cysteine motifs. In the so-called alpha-chemokines, for example, the first two of four cysteines (starting from the amino terminus) are separated by an intervening amino acid (i.e., having the motif C—X—C). The beta-chemokines are characterized by the absence of an intervening amino acid between first two cysteines (i.e., comprising the motif C—C). The smaller gamma- and delta- chemokine families are characterized by a single C residue (gamma) or a pair of cysteines separated by three residues (delta; i.e., comprising the motif CX₃C). For a recent review on chemokines, see Ward et al., 1998, Immunity 9:1-11; and Baggiolini et al., 1998, Nature 392:565-568, and the references cited therein.

Chemokine activity can be mediated by receptors. For example, several seven-transmembrane-domain G protein-coupled receptors for C—C chemokines have been cloned, including: a C—C chemokine receptor-1 which recognizes MIP-1α, RANTES, MCP-2, MCP-3, and MIP-5 (Neote et al., 1993, Cell, 72:415-415); CCR2 which is a receptor for MCP1, 2, 3 and 4 or 5; CCR3 which is a receptor for RANTES, MCP-2, 3, 4, MIP-5 and eotaxin; CCR5 which is a receptor for MIP-1α, MIP-1β and RANTES; CCR4 which is a receptor for MDC or TARC; CCR6 which is a receptor for LARC; and CCR7 which is a receptor for SLC and ELC (MIP-3β; reviewed in Sallusto et al., 1998, Immunol. Today 19:568 and Ward et al., 1998, Immunity 9:1-11). These chemokine receptors typically exist in a inactive form and become activated and transduce a signal once bound by a ligand (i.e., a chemokine).

Another chemokine receptor termed CCX CKR (CCX chemokine receptor) has recently been identified and the gene sequence and corresponding protein sequence determined. This receptor has high affinity for the chemokines SLC (also sometimes referred to as 6-Ckine), ELC (also sometimes referred to as MIP-3β) and TECK. See, e.g., WO 01/27146, which is incorporated herein by reference in its entirety for all purposes.

### SUMMARY

Various methods are provided that are based in part upon unique features of the chemokine receptor, CCX CKR, that are described herein, including: 1) expression of the receptor in a variety of tissue types instead of just cells involved in cellular migration, 2) limited activity for several activities common to most chemokine receptors aspects, 3) autoactivity, whereas most chemokine receptors are activated only upon ligand binding, and 4) involvement in cell growth rather than simply cell migration.

Certain methods that are provided are designed to screen for modulators of cell proliferation. Some of these methods involve contacting a population of cells that comprise a recombinant construct encoding CCX CKR with a test compound, and then determining the effect of the test compound on proliferation of the cell population. In some instances, the contacting step is carried out in the absence of a ligand that modulates CCX CKR activity. The effect of the test compound on proliferation can be determined in a number of different ways, such as 1) counting the number of cells in the cell population using a hemacytometer at a plurality of time points, 2) conducting an assay that measures the metabolic activity of the population of cells, 3) conducting an assay that measures DNA replication in the population of cells, and 4) conducting an assay that measures the concentration of a cell cycle antigen in the population of cells, wherein the antigen is specific to proliferating cells.

Other screening methods that are provided are to identify a modulator of an activity of CCX chemokine receptor (CCX CKR). These methods can involve contacting a cell comprising a recombinant construct encoding CCX CKR with a test compound in the absence of a ligand that specifically binds CCX CKR, and then determining the effect of the test compound on the CCX CKR activity.

Another class of methods that are disclosed herein are for stimulating cell growth. These methods generally involve activating the expression and/or activity of CCX CKR in a cell. In some instances, activation is achieved by introducing a nucleic acid construct encoding CCX CKR into the cell, whereby expression of CCX CKR in the cell is increased. Activation can be performed in the absence of a ligand that specifically binds CCX CKR. The methods can be performed in vivo, in vitro and ex vivo.

Certain cell growth methods involve activating expression and/or activity of CCX CKR in a population of cells, whereby a tissue is formed. The cell population can be of varying types, including bone marrow cells or endothelial cells, for example. Some methods further include transplanting the tissue to a patient.

Methods for treating a disease associated with cell proliferation are also provided. Certain of these methods involve administering to a patient having or susceptible to the disease a compound that inhibits the cell proliferative activity of CCX chemokine receptor (CCX CKR). Exemplary diseases that can be treated include leukemia, kidney or liver cancer, and brain tumors.

### BRIEF DESCRIPTION OF THE DRAWINGS

FIG. 1 shows cells and tissues that express CCX CKR RNA, as determined by RT-PCR of cytoplasmic RNA from cultured primary cells and whole tissues from the organs as indicated.

FIG. 2 is a chart showing changes in protein expression levels under different conditions as determined by PowerBlot™ analysis. Differences in protein expression levels for untransfected human embryonic kidney (HEK) 293 cells and HEK293 cells transformed with CCX CKR, both in the presence and absence of CCX CKR ligands are shown. The sample numbers refer to the following conditions: 1) protein expression in untransfected HEK293 cells, 2) HEK293 cells in the presence of ELC, 3) cells transformed with CCX CKR but no ligand, 4) cells transformed with CCX CKR in the presence of ELC, 5) cells transformed with CCX CKR in the presence of SLC, and 6) cells transformed with CCX CKR in the presence of TECK.

The chart illustrates that simply transfecting human embryonic kidney (HEK) 293 cells with CCX CKR results in a significant alteration in protein expression relative to non-transfected cells, with the expression of a number of proteins increasing and the expression of a number of other proteins decreasing. The chart also shows that the addition of a CCX CKR ligand such as ELC, SLC or TECK to cells transformed with CCX CKR had only a modest effect on protein expression relative to cells only transformed with CCX CKR but not contacted with ligands.

FIG. 3 is a chart that illustrates the effect that transforming cells with CCX CKR has on cell growth. Results are shown for a polyclonal population of transformed HEK293 cells, as well as individual clones obtained by a limiting dilution of the polyclonal transformants (i.e., the cells have a single gene copy). The chart demonstrates that cells in the polyclonal transformant population and individual clones exhibit a significant increase in cell growth relative to non-transformed cells.

FIG. 4 is a chart of growth curves for untransfected HEK293 cells, HEK293 cells transfected with CCR6 (another chemokine receptor) and HEK293 cells transfected with CCX CKR over a 6 day period, both in the presence and absence of the chemokine TECK. The results on the chart illustrate that transfecting HEK293 cells with CCR6 had little effect on cell growth. In contrast, transforming identical cells with CCX CKR resulted in a significant increase in cell proliferation, even in the absence of ligand. The addition of the chemokine ligand TECK to CCX CKR-transformed HEK293 cells had only a minor effect on cell growth compared to simply transforming HEK293 cells with CCX CKR.

FIG. 5 is a plot showing another set of cell growth curves over a four day period. The chart depicts results of a control experiment conducted to confirm that the significant increase in cell growth observed with CCX CKR-transformed cells was not a spurious result due to the presence of a CCX CKR ligand in the serum contained in the cell growth medium. Growth curves for untransformed and transformed HEK293 cells are shown for cell cultures grown in the absence of serum. In both sets of cells, cell growth decreases over time due to the lack of serum (i.e., the cells essentially starve), but cell growth is nonetheless initially significantly higher for the CCX CKR transformed cells.

### DETAILED DESCRIPTION

I. Definitions

The term “CCX CKR” as used herein generally refers to a protein having a native CCX CKR amino acid sequence, as well as variants, fragments and modified forms regardless of origin or mode of preparation. The term thus encompasses both naturally occurring and recombinant forms of CCX CKR. The term includes CCX CKR isolated from natural sources, CCX CKR produced by synthetic methods that are well known in the art, and/or by recombinant or transgenic means. “Naturally occurring CCX CKR” or simply “native CCX CKR” refers to CCX CKR forms that can be isolated from natural sources. The term naturally occurring CCX CKR specifically encompass naturally occurring truncated or soluble forms, naturally occurring variant forms (e.g., alternatively spliced forms), naturally occurring allelic variants of CCX CKR and forms including postranslational modifications (e.g., glycosylation and sulfonation). One specific example of a naturally occurring CCX CKR is described in PCT publication WO 01/27146.

“Recombinant CCX CKR” (rCCX CKR) refers to CCX CKR produced using genetic engineering, recombinant or transgenic techniques. Recombinant CCX CKR forms can be glycosylated or unglycosylated depending upon the organism in which the enzyme is expressed. The term CCX CKR can include both human forms of CCX CKR, as well as other mammalian CCX CKRs (e.g., those from primates such as monkeys, chimpanzee and gorilla, as well as non-primates such as mice, rabbits, cows and swine).

“CCX CKR variants” refer to proteins that are functional equivalents to a native sequence CCX CKR protein and that have similar amino acid sequences and retain, to some extent, one or more of the activities of naturally occurring CCX CKR. CCX CKR activities include, but are not limited to, capacity to bind SLC, ELC, or TECK, typically all three. Other exemplary CCX CKR activities include capacity to stimulate cell growth in certain cell types as described in detail herein.

Variants also include fragments that retain CCX CKR activity. Fragments include the active site of CCX CKR and typically include at least 5-20 flanking amino acids on either side of the active site. Fragments usually include at least 25, 50, 75, 100, 150, 200, 250, or 300 amino acids.

CCX CKR variants also include proteins that are substantially identical (i.e., have substantial sequence identity—see below) to a native sequence of CCX CKR. Such variants include proteins having amino acid alterations such as deletions, insertions and/or substitutions. A “deletion” refers to the absence of one or more amino acid residues in the related protein. The term “insertion” refers to the addition of one or more amino acids in the related protein. A “substitution” refers to the replacement of one or more amino acid residues by another amino acid residue in the polypeptide. Typically, such alterations are conservative in nature such that the activity of the variant protein is substantially similar to a native sequence for CCX CKR (see, e.g., Creighton (1984) Proteins, W.H. Freeman and Company). In the case of substitutions, the amino acid replacing another amino acid usually has similar structural and/or chemical properties. Insertions and deletions are typically in the range of 1 to 5 amino acids, although depending upon the location of the insertion, more amino acids can be inserted or removed. The variations can be made using methods known in the art such as site-directed mutagenesis (Carter, et al. (1986) Nucl. Acids Res. 13:4331; Zoller et al. (1987) Nucl. Acids Res. 10:6487), cassette mutagenesis (Wells et al. (1985) Gene 34:315), restriction selection mutagenesis (Wells, et al. (1986) Philos. Trans. R. Soc. London SerA 317:415), and PCR mutagenesis (Sambrook, et al. (1989) Molecular Cloning, Cold Spring Harbor Laboratory Press).

CCX CKR variants also include modified or derivative forms of CCX CKR. Modified CCX CKR generally refers to proteins in which one or more amino acids of a native sequence CCX CKR have been altered to a non-naturally occurring amino acid residue. Such modifications can occur during or after translation and include, but are not limited to, phosphorylation, glycosylation, sulfonation, cross-linking, acylation and proteolytic cleavage.

“Cell proliferation” refers to a measurement of the number of cells that are dividing in a sample (e.g., a culture).

The terms “modulator” and “modulation” of chemokine receptor activity, as used herein in its various forms, is intended to encompass antagonism, agonism, partial antagonism and/or partial agonism of the activity associated with CCX CKR. In various embodiments, “modulators” may inhibit or stimulate CCX CKR expression or activity.

The terms “nucleic acid” and “polynucleotide” are used interchangeably and refer to deoxyribonucleotides or ribonucleotides and polymers thereof in either single-or double-stranded form. Unless specifically limited, the disclosure of a polynucleotide sequence is also intended to refer to the complementary sequence. As used herein, the term “polynucleotide” includes oligonucleotides.

The term “operably linked” refers to a functional relationship between two or more polynucleotide (e.g., DNA) segments: for example, a promoter or enhancer is operably linked to a coding sequence if it stimulates the transcription of the sequence in an appropriate host cell or other expression system. Generally, sequences that are operably linked are contiguous, and in the case of a signal sequence both contiguous and in reading phase. However, enhancers need not be located in close proximity to the coding sequences whose transcription they enhance.

A “recombinant expression cassette,” “expression cassette” or “expression construct” is a nucleic acid construct, generated recombinantly or synthetically, that has control elements that are capable of effecting expression of a gene (e.g., the gene encoding CCX CKR) that is operatively linked to the control elements in hosts compatible with such sequences. Expression cassettes include at least promoters and optionally, transcription termination signals. Typically, the recombinant expression cassette includes at least a nucleic acid to be transcribed (e.g., a nucleic acid encoding a desired polypeptide) and a promoter. Additional factors necessary or helpful in effecting expression can also be used. For example, an expression cassette can also include transcription termination signals, enhancers, and other nucleic acid sequences that influence gene expression.

The term “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. Thus, a “recombinant” polynucleotide is defined either by its method of production or its structure. In reference to its method of production, the process is use of recombinant nucleic acid techniques, e.g., involving human intervention in the nucleotide sequence, typically selection or production. Alternatively, it can be a polynucleotide made by generating a sequence comprising fusion of two fragments which are not naturally contiguous to each other, but is meant to exclude products of nature. Thus, for example, products made by transforming cells with any non-naturally occurring vector is encompassed, as are polynucleotides comprising sequence derived using any synthetic oligonucleotide process. Similarly, a “recombinant” polypeptide is one expressed from a recombinant polynucleotide.

The terms “allele” or “allelic sequence,” as used herein, refer to a naturally-occurring alternative form of a gene encoding the CCX CKR polypeptide (i.e., a polynucleotide encoding an CCX CKR polypeptide). Alleles result from mutations (i.e., changes in the nucleic acid sequence), and sometimes produce altered and/or differently regulated mRNAs or polypeptides whose structure and/or function may or may not be altered. Common mutational changes that give rise to alleles are generally ascribed to natural deletions, additions, or substitutions of nucleotides that may or may not affect the encoded amino acids. Each of these types of changes may occur alone, in combination with the others, or one or more times within a given gene, chromosome or other cellular polynucleotide. Any given gene may have no, one or many allelic forms. As used herein, the term “allele” refers to either or both a gene or an mRNA transcribed from the gene.

The terms “control elements” or “regulatory sequences” include enhancers, promoters, transcription terminators, origins of replication, chromosomal integration sequences, 5′ and 3′ untranslated regions, with which polypeptides or other biomolecules interact to carry out transcription and translation. For eukaryotic cells, the control sequences will include a promoter and preferably an enhancer, e.g., derived from immunoglobulin genes, SV40, cytomegalovirus, and a polyadenylation sequence, and may include splice donor and acceptor sequences. Depending on the vector system and host utilized, any number of suitable transcription and translation elements, including constitutive and inducible promoters, may be used. When referring to CCX CKR, a promoter other than that naturally associated with the CCX CKR coding sequence can be referred to as a “heterologous” promoter.

The term “antisense sequences” refers to polynucleotides having sequence complementary to a RNA sequence. These terms specifically encompass nucleic acid sequences that bind to mRNA or portions thereof to block transcription of mRNA by ribosomes. Antisense methods are generally well known in the art (see, e.g., PCT publication WO 94/12633, and Nielsen et al., 1991, Science 254:1497; OLIGONUCLEOTIDES AND ANALOGUES, A PRACTICAL APPROACH, edited by F. Eckstein, IRL Press at Oxford University Press (1991); ANTISENSE RESEARCH AND APPLICATIONS (1993, CRC Press)).

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an alpha-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

As used herein, the “substantially sequence identity,” refers to two or more sequences or subsequences that have at least 60%, preferably 80%, most preferably 90%, 95%, 98%, or 99% nucleotide or amino acid residue identity, when compared and aligned for maximum correspondence, as measured using one of the following sequence comparison algorithms or by visual inspection. Two sequences (amino acid or nucleotide) can be compared over their full-length (e.g., the length of the shorter of the two, if they are of substantially different lengths) or over a subsequence such as at least about 50, about 100, about 200, about 500 or about 1000 contiguous nucleotides or at least about 10, about 20, about 30, about 50 or about 100 contiguous amino acid residues.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols In Molecular Biology, Greene Publishing and Wiley-Interscience, New York (supplemented through 1999). Each of these references and algorithms is incorporated by reference herein in its entirety. When using any of the aforementioned algorithms, the default parameters for “Window” length. gap penalty, etc., are used.

One example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the first polypeptide (e.g., a polypeptide encoded by the first nucleic acid) is immunologically cross reactive with the second polypeptide (e.g., a polypeptide encoded by the second nucleic acid). Thus, a polypeptide is typically substantially identical to a second polypeptide, for example, where the two peptides differ only by conservative substitutions.

Another indication that two nucleic acid sequences are substantially identical is that the two molecules hybridize to each other under stringent conditions. Substantial identity exists when the segments will hybridize under stringent hybridization conditions to a strand, or its complement, typically using a sequence of at least about 50 contiguous nucleotides derived from the probe nucleotide sequences.

“Stringent hybridization conditions” refers to conditions in a range from about 5° C. to about 20° C. or 25° C. below the melting temperature (Tm) of the target sequence and a probe with exact or nearly exact complementarity to the target. As used herein, the melting temperature is the temperature at which a population of double-stranded nucleic acid molecules becomes half-dissociated into single strands. Methods for calculating the Tm of nucleic acids are well known in the art (see, e.g., Berger and Kimmel, 1987, Methods In Enzymology, Vol. 152: Guide To Molecular Cloning Techniques, San Diego: Academic Press, Inc. and Sambrook et al.; supra; (1989) Molecular Cloning: A Laboratory Manual, 2nd Ed., Vols. 1-3, Cold Spring Harbor Laboratory). As indicated by standard references, a simple estimate of the Tm value may be calculated by the equation: Tm=81.5+0.41(% G+C), when a nucleic acid is in aqueous solution at 1 M NaCl (see e.g., Anderson and Young, “Quantitative Filter Hybridization” in Nucleic Acid Hybridization (1985)). Other references include more sophisticated computations which take structural as well as sequence characteristics into account for the calculation of Tm. The melting temperature of a hybrid (and thus the conditions for stringent hybridization) is affected by various factors such as the length and nature (DNA, RNA, base composition) of the probe and nature of the target (DNA, RNA, base composition, present in solution or immobilized, and the like), and the concentration of salts and other components (e.g., the presence or absence of formamide, dextran sulfate, polyethylene glycol). The effects of these factors are well known and are discussed in standard references in the art, see e.g., Sambrook, supra, and Ausubel, supra. Typically, stringent hybridization conditions are salt concentrations less than about 1.0 M sodium ion, typically about 0.01 to 1.0 M sodium ion at pH 7.0 to 8.3, and temperatures at least about 30° C. for short probes (e.g., 10 to 50 nucleotides) and at least about 60° C. for long probes (e.g., greater than 50 nucleotides). As noted, stringent conditions may also be achieved with the addition of destabilizing agents such as formamide, in which case lower temperatures may be employed.

The term “polypeptide” is used interchangeably herein with the term “protein,” and refers to a polymer composed of amino acid residues linked by amide linkages, including synthetic, naturally-occurring and non-naturally occurring analogs thereof (amino acids and linkages). Peptides are examples of polypeptides.

The term “conservative substitution,” when describing a polypeptide, refers to a change in the amino acid composition of the polypeptide that does not substantially alter the activity of the polypeptide, i.e., substitution of amino acids with other amino acids having similar properties such that the substitutions of even critical amino acids does not substantially alter activity. Conservative substitution tables providing functionally similar amino acids are well known in the art. The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W) (see also, Creighton, 1984, Proteins, W.H. Freeman and Company).

In addition to the above-defined conservative substitutions, other modification of amino acid residues can result in “conservatively modified variants.” For example, one may regard all charged amino acids as substitutions for each other whether they are positive or negative. In addition, conservatively modified variants can also result from individual substitutions, deletions or additions which alter, add or delete a single amino acid or a small percentage of amino acids, e.g., often less than 5%, in an encoded sequence. Further, a conservatively modified variant can be made from a recombinant polypeptide by substituting a codon for an amino acid employed by the native or wild-type gene with a different codon for the same amino acid.

The terms “peptidomimetic” and “mimetic” refer to a synthetic chemical compound that has substantially the same structural and functional characteristics of the CCX CKR polypeptides of the invention. Peptide analogs are commonly used in the pharmaceutical industry as non-peptide drugs with properties analogous to those of the template peptide. These types of non-peptide compound are termed “peptide mimetics” or “peptidomimetics” (Fauchere, J. Adv. Drug Res. 15:29 (1986); Veber and Freidinger TINS p. 392 (1985); and Evans et al. J. Med. Chem. 30:1229 (1987), which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as a CCX CKR, but have one or more peptide linkages optionally replaced by a linkage selected from the group consisting of, e.g., —CH2NH—, —CH2S—, —CH2—CH2—, —CH═CH— (cis and trans), —COCH2—, —CH(OH)CH2—, and —CH2SO—. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. For example, a mimetic composition is within the scope of the invention if it is capable of carrying out the binding or enzymatic activities of CCX CKR.

By “pharmaceutically acceptable” it is meant the carrier, diluent or excipient must be compatible with the other ingredients of the formulation and not deleterious to the recipient thereof.

As used herein, a receptor -mediated “biological effect” refers to a change in cell function or structure that results from the binding of the receptor to a naturally occurring ligand, (e.g., CCX CKR binding of ELC) and can include receptor internalization, receptor-mediated signaling (e.g., activation of a mammalian G protein, induction of rapid and transient increase in the concentration of cytosolic free calcium), a cellular response function (e.g., induction of cell growth, stimulation of chemotaxis, or release of inflammatory mediators), and the like.

The term “cell proliferative disorder” includes disorders involving the undesired proliferation of a cell or a disorder associated with insufficient cellular proliferation such that increased levels of cellular proliferation are desired (e.g., enhancing angiogenesis). Non-limiting examples of such disorders include tumors, (e.g., brain, lung (small cell and non-small cell), ovary, prostate, breast or colon) or other carcinomas or sarcomas (e.g., leukemia, lymphoma).

The term “angiogenesis” broadly refers to the process of developing new blood vessels. The process involves proliferation, migration and tissue infiltration of capillary endothelial cells from pre-existing blood vessels. Angiogenesis is important in normal physiological processes, including for example, follicular growth, embryonal development and wound healing and in pathological processes such as tumor growth and metastasis. Modulation of angiogenesis refers to both an increase or decrease in the development of blood vessels.

By an “effective” amount (or “therapeutically effective” amount) of a pharmaceutical composition is meant a sufficient, but nontoxic amount of the agent to provide the desired effect. The term refers to an amount sufficient to treat a subject. Thus, the term therapeutic amount refers to an amount sufficient to remedy a disease state or symptoms, by preventing, hindering, retarding or reversing the progression of the disease or any other undesirable symptoms whatsoever. The term prophylactically effective amount refers to an amount given to a subject that does not yet have the disease, and thus is an amount effective to prevent, hinder or retard the onset of a disease.

The term “antibody” as used herein includes antibodies obtained from both polyclonal and monoclonal preparations, as well as the following: (i) hybrid (chimeric) antibody molecules (see, for example, Winter et al. (1991) Nature 349:293-299; and U.S. Pat. No. 4,816,567); (ii) F(ab′)2 and F(ab) fragments; (iii) Fv molecules (noncovalent heterodimers, see, for example, Inbar et al. (1972) Proc. Natl. Acad. Sci. USA 69:2659-2662; and Ehrlich et al. (1980) Biochem 19:4091-4096); (iv) single-chain Fv molecules (sFv) (see, for example, Huston et al. (1988) Proc. Natl. Acad. Sci. USA 85:5879-5883); (v) dimeric and trimeric antibody fragment constructs; (vi) humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); (vii) Mini-antibodies or minibodies (i.e., sFv polypeptide chains that include oligomerization domains at their C-termini, separated from the sFv by a hinge region; see, e.g., Pack et al. (1992) Biochem 31:1579-1584; Cumber et al. (1992) J. Immunology 149B:120-126); and, (vii) any functional fragments obtained from such molecules, wherein such fragments retain specific-binding properties of the parent antibody molecule.

Certain “high affinity” antibodies have an association constant (Ka) of at least about 10⁶M⁻¹, preferably at least about 10⁸M⁻¹, more preferably at least about 10⁹M⁻¹ or greater, e.g., up to 10¹²M⁻¹ or greater.

An “agonist antibody” refers to an antibody that can bind to and activate an activity of CCX CKR (e.g., promotion of cell growth). Some agonist antibodies bind to extracellular domains of CCX CKR. Agonist antibodies can be from any of the classes of antibodies defined above.

“Antagonist antibodies” are antibodies that upon binding CCX CKR inhibit an activity of the receptor (e.g., inhibition of cell growth). These antibodies can also bind to an extracellular domain of CCX CKR and can be any of the types of antibodies listed above.

II. Overview

As noted in the background section above, different chemokine receptors are involved in diverse biological activities, such that collectively the receptors have been implicated in a large number of different biological activities. The current inventors have conducted a number of investigations with a particular chemokine receptor, namely CCX CKR, and determined that it has several distinctive features.

First, the receptor is expressed in a variety of different tissue types. Most chemokine receptors, in contrast, are expressed primarily in white blood cells due to the involvement of many chemokine receptors in cell migration. That CCX CKR is expressed in a variety of other cell types indicates that CCX CKR has a role in biological activities other than simply cell migration. Second, the receptor showed low or no activity in assays designed to measure activities common to many chemokine receptors (e.g., ligand-dependent calcium signaling, ligand-dependent inositol phosphate turnover, ligand-dependent metabolic rate using a MDC physiometer, and ligand-dependent chemotaxis). This also suggests that CCX CKR activities include those not usually associated with chemokine receptors. Third, CCX CKR was found to be autoactive, meaning that that the receptor can trigger biological activities, even in the absence of the receptor's cognate ligands (e.g., SLC, ELC and TECK). Thus, expression of the receptor alone is sufficient in some instances to cause a significant biological response. This, too, is contrary to what is observed with many chemokine receptors, which typically are not activated until a ligand binds the receptor. Finally, CCX CKR was found to play an important role in the regulation of cell growth and protein expression. Simply increasing expression of the receptor has a significant effect on the expression level of a number of different proteins and results in a significant increase in cell growth. So unlike some other chemokine receptors that are primarily involved in cell migration, CCX CKR can play a key role in cell growth and in the regulation of gene expression. This is consistent with the finding that CCX CKR is expressed in many cell types.

Based upon these findings, the inventors have developed a variety of different methods for screening for compounds that modulate the activity of CCX CKR. Because of the observation that CCX CKR is autoactive, some of these screening methods can be conducted without introducing a cognate ligand for the receptor, as is required with many other screening assays. Some of these assays can be utilized to identify modulators that affect the ability of CCX CKR to induce cell growth. Other assays are conducted to identify modulators of other activities associated with CCX CKR.

Given the role that CCX CKR plays in cell growth, methods for treating cellular proliferative disorders are also provided. Certain treatment methods are provided for inhibiting cell growth (e.g., treating certain cancers), whereas other methods can be utilized to induce desired cell growth (e.g., angiogenesis in desired regions and bone marrow transplant). Methods for promoting cell growth of cells or tissues in cell culture or development of artificial tissues (e.g., organs) are also disclosed.

III. Screening for Modulators of CCX CKR Activity

A. General

As noted above and as described in greater detail below in Examples 3 and 4, CCX CKR was found to be an autoactive chemokine receptor. The receptor thus exhibits significant basal or constitutive activity, even in the absence of ligands. The results obtained indicate that cognate ligands for CCX CKR such as SLC, ELC and TECK have a role in fine tuning the response of CCX CKR, causing the receptor activity to be increased or decreased somewhat relative to the basal level of activity associated simply with the expression of CCX CKR in a cell. Because CCX CKR is autoactive, compounds that can modulate CCX CKR activity can be identified in the absence of CCX CKR ligands. Instead, test compounds can simply be brought into contact with a cell expressing CCX CKR and the effect on the autoactive activity level determined. The assay composition can thus be substantially free or completely free of CCX CKR ligands such as SLC, ELC and TECK, as these ligands are not required to trigger the activity of the receptor. The term “substantially free” as used with respect to CCX CKR ligand concentrations generally means that the ligand concentration is sufficiently low such that no detectable change in CCX CKR activity is observed relative to a control (e.g., an assay in which ligand is omitted) or that no detectable level of ligand can be detected by conventional detection means (e.g., immunological assay for the ligand). In solutions that are substantially free of CCX CKR ligands, typically the ligand concentration is less than 10 pH, and in other instances less than 1 pH. Assays of this type differ from conventional assays for modulators of chemokine receptors, because typically these receptors are not autoactive. The assays consequently are generally performed in the presence of a ligand for the chemokine receptor. So unlike conventional assays for agonists or antagonists of typical chemokine receptors, assays can be conducted in the absence of receptor ligand.

Because CCX CKR expression levels are important in cell growth for some cell types, some screening assays are conducted to identify compounds that are modulators of cell growth. Such compounds can be candidate compounds for inhibiting cell growth, making the compounds useful in inhibiting undesired cell growth (e.g., cancer or tumor formation). Other screening assays can be conducted to identify compounds that promote the capacity of CCX CKR to induce cellular growth. Such compounds can be utilized to activate desired cell growth (e.g., bone marrow transplants, angiogenesis, and tissue or organ formation). Other activities can be assayed, however. Examples of such activities are cell signaling, pH changes, metabolic rate, kinase cascades, gene expression or cell migration.

Cells utilized in the screening assays can be conducted with cells that naturally express CCX CKR or with cells that have been transformed to express CCX CKR. Such transformants can be prepared using conventional recombinant technologies that are known to those of skill in the art and that have been discussed previously (see, e.g., WO 01/27146). If transformed cells are utilized, the cells are stably or transiently transfected with a vector or expression cassette that encodes CCX CKR. Specific examples of the type of cells that can be utilized include HEK293 cells, CHO cells, BHK cells, HeLa cells, COS cells and the like.

The cells are contacted with the test agent under conditions and for a time sufficient for the test agent to bind to CCX CKR. The effect of the test agent on cell growth is then determined. The effect on cell growth activity is sometimes determined relative to a control activity level that can be a historical control (i.e., an activity level determined prior to the assay with the test agent that can be based on a single determination, but more typically based upon an average or other statistical value) or a control assay that is run concurrently and in parallel with the assay with the test agent. The control can be, for example, an assay run in the absence of the test agent and/or performed with a cell that does not express CCX CKR.

B. Exemplary Cell Proliferation Assays

Cellular proliferation assays can be conducted in a variety of different ways, including, for example: actual cell counting, clonogenic assays, measuring metabolic activity, measuring DNA synthesis and/or measuring the level of molecules that regulate cell cycle (e.g., CDK kinase assays). A brief summary of these approaches follows. For a general review of some of these approaches, see for example, Roche Molecular Biochemicals, “Apoptosis and Cell Proliferation”, 2^{nd} Revised edition, pages 66-114, which is incorporated herein by reference in its entirety for all purposes.

One approach is simply to count the number of cells using a cell counting device such as a hemacytometer (see, e.g., Example 4). In the clonogenic assay approach, a defined number of cells are plated out onto a suitable media and the number of colonies that are formed after a defined period of time are determined. The clonogenic approach can be somewhat cumbersome for large number of samples and for cells that divide only a few times and then become quiescent.

A number of different assays for measuring metabolic activity are available. One approach is to incubate the cells with a tetrazolium salt (e.g., MTT, XTT or WST-1), which becomes cleaved during cellular metabolism to form a colored formazan product. Further guidance regarding assays of this type are provided by Cook, J. A. and Mitchell, J. B. (1989) Anal. Biochem. 179:1; Roehm, N. W. et al. (1991) J. Immunol. Methods 142:257; Slater, T. F., et al. (1963) Biochem. Biophys. Acta 77:383; Berridge, M. V. and Tan, A. S. (1993) Arch. Biochem. Biophys. 303:474; Cory, A. H., et al. (1991) Cancer Commun. 3:207; Jabbar, S. A. B., et al. (1989) Br. J. Cancer 60: 523; and Scudiero, E. A., et al. (1988) Cancer Res. 48, 4827, each of which is incorporated herein by reference in its entirety for all purposes. A variety of kits for performing such assays are available from Roche Molecular Biochemicals. Other assays in this class involve the measurement of ATP and involve detecting the formation of luminescence formed via the activity of luciferase. Such assays are commercially available from Perkin Elmer (see, e.g., ATPlite™ Assay kits).

Because DNA is replicated during cell proliferation, assays that provide a measure of DNA replication also provide an useful measure of cell proliferation. Assays of this type typically involve adding labeled DNA precursors to a cell culture. Cells that are about to divide incorporate the labeled nucleotide into their DNA. Some approaches utilize tritiated thymidine ([3H]-TdR) and measure the amount of incorporated tritiated thymidine using liquid scintillation counting. To avoid using radioactive compounds, other assays utilize the thymidine analog 5-bromo-2′deoxy-uridine (BrdU), which becomes incorporated into DNA just like thymidine. Incorporated BrdU can be detected quantitatively using a cellular immunoassay that utilizes monoclonal antibodies directed against BrdU. Commercial kits for performing such assays are available from a number of sources including Roche Molecular Biochemicals.

Other assays capitalize on the fact that certain cell cycle antigens are specific to proliferating cells. Molecules involved in the regulation of cell cycle can be detected either by their activity or by quantitating their amount (e.g., via Western blots, ELISA or immunohistochemistry). Examples of nuclear antigens present only in proliferating cells that can be measured include, but are not limited to, proliferating cell nuclear antigen (PCNA), Ki-67 and topoisomerase II-alpha (Ki-S1). Kits commercially available to perform such assays are available from various suppliers, including Roche Molecular Biochemicals.

C. Test Agents

A variety of different types of agents can be screened for the ability to modulate CCX CKR activity, either in the presence or absence of CCX CKR ligands. The agents can be agonists or antagonists. The agents can be include, for example, antibodies, peptides or small molecules, hormones, growth factors, cytokines, chemokines, naturally occurring molecules, or molecules from existing repertoires of chemical compounds synthesized by the pharmaceutical industry. Combinatorial libraries can be produced for many types of compounds that can be synthesized in a step-by-step fashion. Such compounds include polypeptides, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described in PCT Publications WO 95/12608, WO 93/06121, WO 94/08051, 95/35503 and WO 95/30642. Peptide libraries can also be generated by phage display methods. See, e.g., Devlin, WO 91/18980. Compounds to be screened can also be obtained from the National Cancer Institute's Natural Product Repository, Bethesda, Md. The agents to be screened can also be agonist antibodies and antagonist antibodies. A general review of methods for preparing libraries is provided by Dolle and Nelson (J. Combinatorial Chemistry 1: 235-282 (1999)).

One class of compounds that can be screened are those that are based upon the structure of the binding domains of the CCX CKR ligands SLC, ELC and TECK. For example synthetic peptides derived from the N-terminus or C-terminus of these ligands may act as modulators of CCX CKR function.

Another class of compounds that can be screened, are mimetics of these polypeptide ligands, agonists and antagonist. The terms “mimetic” and “peptidomimetic” refer to a synthetic chemical compound which has substantially the same structural and/or functional characteristics as a CCX CKR polypeptide ligand, agonist or antagonist. The mimetic can be either entirely composed of synthetic, non-natural analogues of amino acids, or, be a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. Polypeptide mimetic compositions can contain any combination of nonnatural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, New York).

A polypeptide can also be characterized as a mimetic by containing all or some non-natural residues in place of naturally occurring amino acid residues. Nonnatural residues are well described in the scientific and patent literature.

The mimetics can also include compositions that contain a structural mimetic residue, particularly a residue that induces or mimics secondary structures, such as a beta turn, beta sheet, alpha helix structures, gamma turns, and the like. For example, substitution of natural amino acid residues with D-amino acids; N-alpha-methyl amino acids; C-alpha-methyl amino acids; or dehydroamino acids within a peptide can induce or stabilize beta turns, gamma turns, beta sheets or alpha helix conformations. Beta turn mimetic structures have been described, e.g., by Nagai (1985) Tet. Lett. 26:647-650; Feigl (1986) J. Amer. Chem. Soc. 108:181-182; Kahn (1988) J. Amer. Chem. Soc. 110:1638-1639; Kemp (1988) Tet. Lett. 29:5057-5060; Kahn (1988) J. Molec. Recognition 1:75-79. Beta sheet mimetic structures have been described, e.g., by Smith (1992) J. Amer. Chem. Soc. 114:10672-10674. For example, a type VI beta turn induced by a cis amide surrogate, 1,5-disubstituted tetrazol, is described by Beusen (1995) Biopolymers 36:181-200. Incorporation of achiral omega-amino acid residues to generate polymethylene units as a substitution for amide bonds is described by Banerjee (1996) Biopolymers 39:769-777.

The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman et al. (Eds) John Wiley & Sons, Inc., New York. Polypeptides incorporating mimetics can also be made using solid phase synthetic procedures, as described, e.g., by Di Marchi, et al., U.S. Pat. No. 5,422,426. Mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234.

Other compounds are based upon agonists and antagonists for CCX CKR, which have been identified and are shown below:

TABLE 1
Exemplary Small Molecule Modulators of CCX CKR Function

| | |
|---|---|
| Compound I (antagonist) | 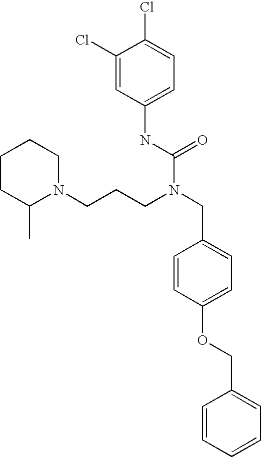 |
| Compound II (antagonist) | 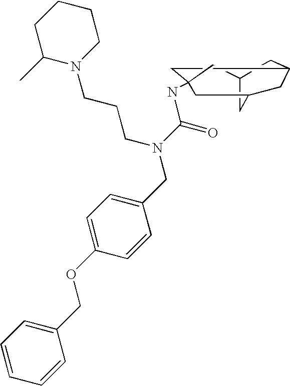 |
| Compound III (agonist) | 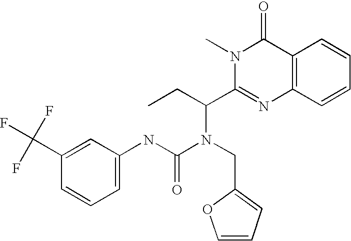 |

Yet another class of compounds that can be screened are antibodies, including agonist and antagonist antibodies. Such antibodies can be prepared according to a number of methods. For instance, for methods for production of polyclonal or monoclonal antibodies, see Coligan, CURRENT PROTOCOLS IN IMMUNOLOGY, Wiley/Greene, New York (1991); Stites et al. (eds.) BASIC AND CLINICAL IMMUNOLOGY (7th ed.) Lange Medical Publications, Los Altos, Calif., and references cited therein (“Stites”); Goding, MONOCLONAL ANTIBODIES: PRINCIPLES AND PRACTICE (2d ed.) Academic Press, New York, N.Y. (1986); Kohler and Milstein, 1975, Nature 256:495-97; and Harlow and Lane. These techniques include antibody preparation by selection of antibodies from libraries of recombinant antibodies in phage or similar vectors. See, Huse et al., 1989, Science 246:1275-81; and Ward et al., 1989, Nature 341:544-46.

For methods of preparing humanized antibodies, see Queen, et al., 1989, Proc. Nat'l Acad. Sci. USA 86:10029; U.S. Pat. Nos. 5,563,762; 5,693,761; 5,585,089 and 5,530,101. The human antibody sequences used for humanization can be the sequences of naturally occurring human antibodies or can be consensus sequences of several human antibodies. See Kettleborough et al., Protein Engineering 4:773 (1991); Kolbinger et al., Protein Engineering 6:971 (1993). Humanized monoclonal antibodies against CCX CKR can also be produced using transgenic animals having elements of a human immune system (see, e.g., U.S. Pat. Nos. 5,569,825; 5,545,806; 5,693,762; 5,693,761; and 5,7124,350).

Antibodies that bind CCX CKR can also be produced using phage display technology (see, e.g., Dower et al., WO 91/17271 and McCafferty et al., WO 92/01047). In these methods, libraries of phage are produced in which members display different antibodies on their outer surfaces. Antibodies are usually displayed as Fv or Fab fragments.

An antibody is substantially pure when at least about 80%, more often at least about 90%, even more often at least about 95%, most often at least about 99% or more of the polypeptide molecules present in a preparation specifically bind the same antigen (e.g., CCX CKR polypeptide). For pharmaceutical uses, anti-CCX CKR immunoglobulins of at least about 90 to 95% homogeneity are preferred, and 98 to 99% or more homogeneity are most preferred.

IV. Modulating Cell Growth/Tissue Generation

By inducing expression of CCX CKR in cells and thereby stimulating cell growth, cells or tissues can be generated for use in therapy. In general, cells expressing CCX CKR are cultured using conventional in vitro cell culture or tissue engineering methods. Various bioreactors that can be utilized in tissue engineering are described for example in U.S. Pat. Nos. 6,642,019; 6,218,182; 6,080,581; 5,888,807; and 5,763,266.

Cells recovered from such bioreactors or cell culture can then be transferred to a patient or introduced into the patient at a site at which tissue regeneration is needed. The transplanted cells can then undergo further propagation and/or differentiation in the patient. Ex vivo cell therapy methods are described for example by Mayhew et al., (1998) Tissue Eng. 4:325-334 and Wakitani et al. (1998) Tissue Eng. 4:429-444 (1998).

Alternatively, the cells can be grown as a patch of tissue, which is then transferred or transplanted to a patient. In such methods, the cells are sometimes grown on a scaffold provides a support for the developing tissue. Exemplary scaffolds that can be utilized in certain applications include, for example, those described in U.S. Pat. Nos. 6,642,363; 6,623,959; 6,586,246; 6,534,560; 6,514,515; 6,454,811; and 6,306,424.

Certain methods involve transforming heterologous cells with a nucleic acid sequence that is suitable for gene therapy. The resulting heterologous cells are then introduced into the patient. Other methods involve introducing an agonist that promotes CCX CKR activity, thereby activating cell growth.

Cells that are grown for use in such therapies can be of a variety of different types. In general the cells are ones in which CCX CKR is naturally expressed. As described in Example 1 below, such cells include, but are not limited to, dendritic cells, spleen cells, lymph cells, and various non-lymphoid cells such as kidney, heart and olfactory epithelium cells.

As one specific example of the utility of tissue generation methods, bone marrow cell growth can be promoted by increasing CCX CKR activity and/or expression. In another example, the growth of heart cells or cardiomyocytes is stimulated by increasing CCX CKR activity and/or expression.

V. Treatment Methods

A. General

The finding that CCX CKR has an important role in cell proliferation in certain cell types means that treatment methods that either promote or inhibit CCX CKR expression and/or activity can be used to regulate cell growth depending upon the desired outcome. The methods thus generally involve administering a therapeutically or prophylactically effective amount of the pharmaceutical composition which promotes or inhibits CCX CKR expression and/or activity to a patient with a cellular proliferative disease.

For instance, if CCX CKR causes unwanted proliferation (e.g., in certain cancers or tumors), then pharmaceutical compositions that contain an agent that inhibits the expression or activity of CCX CKR can be administered. Examples of such inhibitory agents include antagonists (inhibitors) of CCX CKR activity, antisense molecules, ribozymes, triplex polynucleotides, and small inhibitory RNAs (siRNAs), which are described in greater detail below. The antagonists can be either small molecules, polypeptides, polypeptide mimetics, and antagonist antibodies, for example. If on the other hand, CCX CKR cell growth activity is to be increased to promote desired cell growth (e.g., in bone marrow transplants or angiogenesis), then the pharmaceutical composition includes an agent that promotes the expression or activity of CCX CKR. This can be accomplished, for instance, by gene therapy or by administering an agonist. Suitable agonists include, but are not limited to, small molecule agonists, polypeptide agonists and mimetics thereof, and agonist antibodies. The

Specific examples of diseases in which CCX CKR activity or expression is sought to be inhibited include various types of cancer. For instance, given that CCX CKR has been found to be expressed in kidney, heart, liver and brain, cancers or tumors in these regions can be treated using compositions that inhibit CCX CKR activity or expression. Because CCX CKR is also expressed in some types of leukocytes, diseases associated with unwanted cell growth in such cells can also be treated, including Hodgkin's disease, non-Hodgkin's lymphoma, Burkitt's lymphoma, multiple myeloma, and leukemia.

The involvement of CCX CKR in cell growth also means that inhibitory agents can be administered to treat diseases in which angiogenesis and neovascularization play a role in disease (e.g., neoplastic diseases, retinopathy and macular degeneration). In view of evidence that CCX CKR is also highly expressed in the heart in mice (Townson J R, Nibbs R J. (2002), Eur. J. Immunol. 32:1230-41), compositions that inhibit CCX CKR can also be utilized to treat various heart disorders associated with unwanted cellular growth. Certain conditions, such as myocardial infarction will result in loss of heart tissue. Stimulation of CCX CKR expression or function can be used to enhance heart tissue survival and tissue replacement.

When enhanced cell growth is desired, then the composition that is administered contains an agent that promotes the expression or activity of CCX CKR. Specific examples of such situations include tissue repair, wound healing, inducing angiogenesis in those tissues in which CCX CKR is expressed, and growth of bone marrow in bone marrow transplants.

The methods can be used in the treatment of a variety of different animal subjects/patients. Most typically the subject is a human. But in other instances the subject is a non-human primate (e.g., monkey, chimpanzee, gorilla) or another mammal such as a rabbit, cow, rat or mouse). Such animals can also be useful as model systems for human therapy.

B. Inhibiting CCX CKR Activity or Expression

A variety of different types of compounds can be utilized to inhibit CCX CKR activity or expression. This is useful when treatment calls for a reduction in cell growth. Examples of inhibitory agents include, antisense polynucleotides with specificity for CCX CKR, triplex oligo- and polynucleotides, ribozymes and siRNAs. These are discussed in greater detail below.

1. Antisense Polynucleotides

Antisense oligonucleotides and polynucleotides can be used to inhibit expression of the CCX CKR gene, thereby inhibiting cellular growth. Some therapeutic methods of the invention involve the administration of an oligonucleotide that functions to inhibit or stimulate CCX CKR activity under in vivo physiological conditions, and is relatively stable under those conditions for a period of time sufficient for a therapeutic effect. Polynucleotides can be modified to impart such stability and to facilitate targeting delivery of the oligonucleotide to the desired tissue, organ, or cell.

The antisense polynucleotides of the invention comprise an antisense sequence of at least about 10 bases, typically at least 12 or 14, and up to about 3000 contiguous nucleotides that specifically hybridize to a sequence from mRNA encoding CCX CKR or mRNA transcribed from the CCX CKR gene. More often, the antisense polynucleotide of the invention is from about 12 to about 50 nucleotides in length or from about 15 to about 25 nucleotides in length. In general, the antisense polynucleotide should be long enough to form a stable duplex but short enough, depending on the mode of delivery, to administer in vivo, if desired. The minimum length of a polynucleotide required for specific hybridization to a target sequence depends on several factors, such as G/C content, positioning of mismatched bases (if any), degree of uniqueness of the sequence as compared to the population of target polynucleotides, and chemical nature of the polynucleotide (e.g., methylphosphonate backbone, peptide nucleic acid, phosphorothioate), among other factors.

Generally, to assure specific hybridization, the antisense sequence is substantially complementary to the target CCX CKR mRNA sequence. In certain embodiments, the antisense sequence is exactly complementary to the target sequence. The antisense polynucleotides may also include, however, nucleotide substitutions, additions, deletions, transitions, transpositions, or modifications, or other nucleic acid sequences or non-nucleic acid moieties so long as specific binding to the relevant target sequence corresponding to CCX CKR RNA or its gene is retained as a functional property of the polynucleotide.

In one embodiment, the antisense sequence is complementary to relatively accessible sequences of the CCX CKR mRNA (e.g., relatively devoid of secondary structure). This can be determined by analyzing predicted RNA secondary structures using, for example, the MFOLD program (Genetics Computer Group, Madison Wis.) and testing in vitro or in vivo as is known in the art. Another useful method for identifying effective antisense compositions uses combinatorial arrays of oligonucleotides (see, e.g., Milner et al., 1997, Nature Biotechnology 15:537).

The invention also provides an antisense polynucleotide that has sequences in addition to the antisense sequence (i.e., in addition to anti-CCX CKR-sense sequence). In this case, the antisense sequence is contained within a polynucleotide of longer sequence. In another embodiment, the sequence of the polynucleotide consists essentially of, or is, the antisense sequence.

The antisense nucleic acids (DNA, RNA, modified, analogues, and the like) can be made using any suitable method for producing a nucleic acid, such as the chemical synthesis and recombinant methods disclosed herein. In one embodiment, for example, antisense RNA molecules of the invention may be prepared by de novo chemical synthesis or by cloning. For example, an antisense RNA that hybridizes to CCX CKR mRNA can be made by inserting (ligating) an CCX CKR DNA sequence in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention. The antisense oligonucleotides of the invention can be used to inhibit CCX CKR activity in cell-free extracts, cells, and animals, including mammals and humans.

For general methods relating to antisense polynucleotides, see ANTISENSE RNA AND DNA, (1988), D. A. Melton, Ed., Cold Spring Harbor Laboratory, Cold Spring Harbor, N.Y.). See also, Dagle et al., 1991, Nucleic Acids Research, 19:1805. For a review of antisense therapy, see, e.g., Uhlmann et al., Chem. Reviews, 90:543-584 (1990).

2. Triplex Oligo- and Polynucleotides

The present invention provides oligo- and polynucleotides (e.g., DNA, RNA, PNA or the like) that bind to double-stranded or duplex CCX CKR nucleic acids (e.g., in a folded region of the CCX CKR RNA or in the CCX CKR gene), forming a triple helix-containing, or “triplex” nucleic acid. Triple helix formation results in inhibition of CCX CKR expression by, for example, preventing transcription of the CCX CKR gene, thus reducing or eliminating CCX CKR activity in a cell. Without intending to be bound by any particular mechanism, it is believed that triple helix pairing compromises the ability of the double helix to open sufficiently for the binding of polymerases, transcription factors, or regulatory molecules to occur.

Triplex oligo- and polynucleotides of the invention are constructed using the base-pairing rules of triple helix formation (see, e.g., Cheng et al., 1988, J. Biol. Chem. 263: 15110; Ferrin and Camerini-Otero, 1991, Science 354:1494; Ramdas et al., 1989, J. Biol. Chem. 264:17395; Strobel et al., 1991, Science 254:1639; and Rigas et al., 1986, Proc. Natl. Acad. Sci. U.S.A. 83: 9591; each of which is incorporated herein by reference) and the CCX CKR mRNA and/or gene sequence. Typically, the triplex-forming oligonucleotides of the invention comprise a specific sequence of from about 10 to at least about 25 nucleotides or longer “complementary” to a specific sequence in the CCX CKR RNA or gene (i.e., large enough to form a stable triple helix, but small enough, depending on the mode of delivery, to administer in vivo, if desired). In this context, “complementary” means able to form a stable triple helix. In one embodiment, oligonucleotides are designed to bind specifically to the regulatory regions of the CCX CKR gene (e.g., the CCX CKR 5′-flanking sequence, promoters, and enhancers) or to the transcription initiation site, (e.g., between −10 and +10 from the transcription initiation site). For a review of recent therapeutic advances using triplex DNA, see Gee et al., in Huber and Carr, 1994, MOLECULAR AND IMMUNOLOGIC APPROACHES, Futura Publishing Co, Mt Kisco N.Y. and Rininsland et al., 1997, Proc. Natl. Acad. Sci. USA 94:5854, which are both incorporated herein by reference.

3. Ribozymes

The present invention also provides ribozymes useful for inhibition of CCX CKR activity. The ribozymes of the invention bind and specifically cleave and inactivate CCX CKR mRNA. Useful ribozymes can comprise 5′- and 3′-terminal sequences complementary to the CCX CKR mRNA and can be engineered by one of skill on the basis of the CCX CKR mRNA sequence disclosed herein (see PCT publication WO 93/23572, supra). Ribozymes of the invention include those having characteristics of group I intron ribozymes (Cech, 1995, Biotechnology 13:323) and others of hammerhead ribozymes (Edgington, 1992, Biotechnology 10:256).

Ribozymes of the invention include those having cleavage sites such as GUA, GUU and GUC. Other optimum cleavage sites for ribozyme-mediated inhibition of CCX CKR activity in accordance with the present invention include those described in PCT publications WO 94/02595 and WO 93/23569, both incorporated herein by reference. Short RNA oligonucleotides between 15 and 20 ribonucleotides in length corresponding to the region of the target CCX CKR gene containing the cleavage site can be evaluated for secondary structural features that may render the oligonucleotide more desirable. The suitability of cleavage sites may also be evaluated by testing accessibility to hybridization with complementary oligonucleotides using ribonuclease protection assays, or by testing for in vitro ribozyme activity in accordance with standard procedures known in the art.

As described by Hu et al., PCT publication WO 94/03596, incorporated herein by reference, antisense and ribozyme functions can be combined in a single oligonucleotide. Moreover, ribozymes can comprise one or more modified nucleotides or modified linkages between nucleotides, as described above in conjunction with the description of illustrative antisense oligonucleotides of the invention.

In one embodiment, the ribozymes of the invention are generated in vitro and introduced into a cell or patient. In another embodiment, gene therapy methods are used for expression of ribozymes in a target cell ex vivo or in vivo.

4. Double Stranded RNA Inhibition/siRNA

Double stranded RNA (dsRNA) inhibition methods can also be use to inhibit expression of CCX CKR. The RNA utilized in such methods is designed such that a least a region of the dsRNA is substantially identical to a region of the CCX CKR gene; in some instances, the region is 100% identical to the CCX CKR gene. For use in mammals, the dsRNA is typically about 19-30 nucleotides in length (i.e., small inhibitory RNAs are utilized (siRNA)). Methods and compositions useful for performing dsRNAi and siRNA are discussed, for example, in PCT Publications WO 98/53083; WO 99/32619; WO 99/53050; WO 00/44914; WO 01/36646; WO 01/75164; WO 02/44321; and published U.S. patent application Ser. No. 10/195,034, each of which is incorporated herein by reference in its entirety for all purposes.

5. Antagonist Antibodies

Antibodies having binding specificity for CCX CKR that interferes its activity (e.g., inhibit ligand binding) can also be utilized to inhibit gene protein activity. Such antibodies can be generated from full-length proteins or fragments thereof according to the methods described below. Methods for preparing antibodies that specifically bind to CCX CKR have been described previously in PCT publication WO 01/27146 and are also described infra.

C. Promoting CCX CKR Activity and/or Expression

In instances in which cell growth is a desired outcome, promoting CCX CKR activity or expression levels in tissues in which it is expressed can result in beneficial cell growth. These goals can be achieved, for example, by increasing the level of expression of CCX CKR or CCX CKR protein levels. In one approach, a CCX CKR protein in the form of a pharmaceutical composition such as that described below is administered to an individual. Alternatively, the activity of CCX CKR can also be increased even without an increase in expression levels by introducing a pharmaceutical composition that includes an agonist of CCX CKR. Such an agonist may be a small molecule, a polypeptide, a mimetic or an agonist antibody.

D. Gene Therapy

Gene therapy is another option for either increasing or decreasing CCX CKR expression levels. Gene therapy refers to the introduction of an otherwise exogenous polynucleotide which produces a medically useful phenotypic effect upon the (typically) mammalian cell(s) into which it is transferred. In some instances, gene therapy involves introducing into a cell a vector that: (i) expresses an CCX CKR gene product to increase CCX CKR activity; (ii) expresses a nucleic acid having an CCX CKR gene or mRNA sequence (such as an antisense RNA, e.g., to reduce CCX CKR activity), (iii) expresses a polypeptide or polynucleotide that otherwise affects expression of CCX CKR gene products (e.g., a ribozyme directed to CCX CKR mRNA to reduce CCX CKR activity), or (iv) replaces or disrupts an endogenous CCX CKR sequence (e.g., gene replacement and gene knockout, respectively).

Vectors useful in CCX CKR gene therapy can be viral or nonviral, and include those described supra in relation to the CCX CKR expression systems of the invention. It will be understood by those of skill in the art that gene therapy vectors may comprise promoters and other regulatory or processing sequences (see, e.g., WO 01/27146). Usually the vector comprises a promoter and, optionally, an enhancer (separate from any contained within the promoter sequences) that serve to drive transcription of an oligoribonucleotide, as well as other regulatory elements that provide for episomal maintenance or chromosomal integration and for high-level transcription, if desired. A plasmid useful for gene therapy can comprise other functional elements, such as selectable markers, identification regions, and other sequences. The additional sequences can have roles in conferring stability both outside and within a cell, targeting delivery of CCX CKR nucleotide sequences (sense or antisense) to a specified organ, tissue, or cell population, mediating entry into a cell, mediating entry into the nucleus of a cell and/or mediating integration within nuclear DNA. For example, aptamer-like DNA structures, or other protein binding moieties sites can be used to mediate binding of a vector to cell surface receptors or to serum proteins that bind to a receptor thereby increasing the efficiency of DNA transfer into the cell. Other DNA sites and structures can directly or indirectly bind to receptors in the nuclear membrane or to other proteins that go into the nucleus, thereby facilitating nuclear uptake of a vector. Other DNA sequences can directly or indirectly affect the efficiency of integration.

Suitable gene therapy vectors may, or may not, have an origin of replication. For example, it is useful to include an origin of replication in a vector for propagation of the vector prior to administration to a patient. However, the origin of replication can often be removed before administration if the vector is designed to integrate into host chromosomal DNA or bind to host mRNA or DNA.

As noted, the present invention also provides methods and reagents for gene replacement therapy (i.e., replacement by homologous recombination of an endogenous CCX CKR gene with a recombinant gene). Vectors specifically designed for integration by homologous recombination may be used. Important factors for optimizing homologous recombination include the degree of sequence identity and length of homology to chromosomal sequences. The specific sequence mediating homologous recombination is also important, because integration occurs much more easily in transcriptionally active DNA. Methods and materials for constructing homologous targeting constructs are described by e.g., Mansour et al., 1988, Nature 336: 348; Bradley et al., 1992, Bio/Technology 10: 534. See also, U.S. Pat. Nos. 5,627,059; 5,487,992; 5,631,153; and 5,464,764. In one embodiment, gene replacement therapy involves altering or replacing all or a portion of the regulatory sequences controlling expression of the CCX CKR gene that is to be regulated.

The invention also provides methods and reagents for CCX CKR “gene knockout” (i.e., deletion or disruption by homologous recombination of an endogenous CCX CKR gene using a recombinantly produced vector). In gene knockout, the targeted sequences can be regulatory sequences (e.g., the CCX CKR promoter), or RNA or protein coding sequences. The use of homologous recombination to alter expression of endogenous genes is described in detail in U.S. Pat. No. 5,272,071, WO 91/09955, WO 93/09222, WO 96/29411, WO 95/31560, and WO 91/12650. See also, Moynahan et al., 1996, Hum. Mol. Genet. 5:875.

Gene therapy vectors may be introduced into cells or tissues in vivo, in vitro or ex vivo. For ex vivo therapy, vectors may be introduced into cells, e.g., stem cells, taken from the patient and clonally propagated for autologous transplant back into the same patient (see, e.g., U.S. Pat. Nos. 5,399,493 and 5,437,994, the disclosures of which are herein incorporated by reference).

VI. Pharmaceutical Compositions

A. Composition and Mode of Administration

Therapeutic compositions comprising agonists or antagonists of CCX CKR cellular proliferation active are also provided. These can be utilized in the treatment methods that are described herein to appropriately modulating the cell growth activity associated with CCX CKR.

The agonists or antagonists can be directly administered under sterile conditions to the host to be treated. However, while it is possible for the active ingredient to be administered alone, it is often preferable to present it as a pharmaceutical formulation. Formulations typically comprise at least one active ingredient together with one or more acceptable carriers thereof. Each carrier should be both pharmaceutically and physiologically acceptable in the sense of being compatible with the other ingredients and not injurious to the patient. For example, the bioactive agent can be complexed with carrier proteins such as ovalbumin or serum albumin prior to their administration in order to enhance stability or pharmacological properties such as half-life. Furthermore, therapeutic formulations of this invention can be combined with or used in association with other chemotherapeutic or chemopreventive agents.

Therapeutic formulations can be prepared by any methods well known in the art of pharmacy. See, e.g., Gilman et al (eds.) (1990) Goodman and Gilman's: The Pharmacological Bases of Therapeutics (8th ed.) Pergamon Press; and (1990) Remington's Pharmaceutical Sciences (17th ed.) Mack Publishing Co., Easton, Pa.; Avis et al (eds.) (1993) Pharmaceutical Dosage Forms: Parenteral Medications Dekker, New York; Lieberman et al. (eds.) (1990) Pharmaceutical Dosage Forms: Tablets Dekker, New York; and Lieberman et al (eds.) (1990) Pharmaceutical Dosage Forms: Disperse Systems Dekker, New York.

Depending on the disease to be treated and the subject's condition, the compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. As used herein, topical application is also meant to include the use of mouth washes and gargles.

The pharmaceutical composition and method of the present invention may further comprise other therapeutically active compounds as noted herein which are usually applied in the treatment of the above mentioned pathological conditions.

For modulating angiogenesis, a variety of specific delivery options are available. Depending upon the particular application, the compositions can be targeted to specific areas or tissues of a subject. For example, in some methods, a composition is delivered to specific regions of the heart to treat various disorders such as ischemia. Other treatments, in contrast, involve administering the composition in a general manner without seeking to target delivery to specific regions.

A number of approaches can be utilized to localize the delivery of agents to particular regions. Certain of these methods involve delivery to the body lumen or to a tissue (see, e.g., U.S. Pat. Nos. 5,941,868; 6,067,988; 6,050,986; and 5,997,509; as well as PCT Publications WO 00/25850; WO 00/04928; 99/59666; and 99/38559). Delivery can also be effectuated by intramyocardial injection or administration. Examples of such approaches include those discussed in U.S. Pat. Nos. 6,086,582; 6,045,565; 6,056,969; and 5,997,525; and in PCT Publications WO 00/16848; WO 00/18462; WO 00/24452; WO 99/49773 and WO 99/49926. Other options for local delivery include intrapericardial injection (see, e.g., U.S. Pat. Nos. 5,931,810; 5,968,010; and 5,972,013) and perivascular delivery. Various transmyocardial revascular (TMR) channel delivery approaches can be utilized as well. Many of these methods utilize a laser to conduct the revascularization. A discussion of such approaches is set forth in U.S. Pat. Nos. 5,925,012; 5,976,164; 5,993,443; and 5,999,678, for example. Other options include intraarterial and/or intracoronary delivery, for example coronary artery injection (see, e.g., WO 99/29251) and endovascular administration (see, e.g., U.S. Pat. Nos. 6,001,350; 6,066,123; and 6,048,332; and PCT Publications WO 99/31982; WO 99/33500; and WO 00/15285). Thus, for example, one can inject a composition as described herein directly into the myocardium.

Additional options for the delivery of compositions to modulate angiogenesis include systemic administration using intravenous or subcutaneous administration, cardiac chamber access (see, e.g., U.S. Pat. No. 5,924,424) and tissue engineering (U.S. Pat. No. 5,944,754).

Other delivery methods known by those skilled in the art include the methods disclosed in U.S. Pat. Nos. 5,698,531; 5,893,839; 5,797,870; 5,693,622; 5,674,722; 5,328,470; and 5,707,969.

B. Dosage

In the treatment or prevention of conditions which require modulation of CCX CKR growth activity, an appropriate dosage level will generally be about 0.001 to 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be about 0.005 to about 0.05, 0.05 to 0.5 or 0.5 to 5 mg/kg per day. For oral administration, the compositions are preferably provided in the form of tablets containing about 1 to 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The following examples are provided to illustrate in greater detail certain aspects of the compositions and methods that are provided. These examples, however, should not be construed to limit the currently claimed invention.

### EXAMPLE 1

### Expression of CCX CKR in Leukocytes and Various Tissues

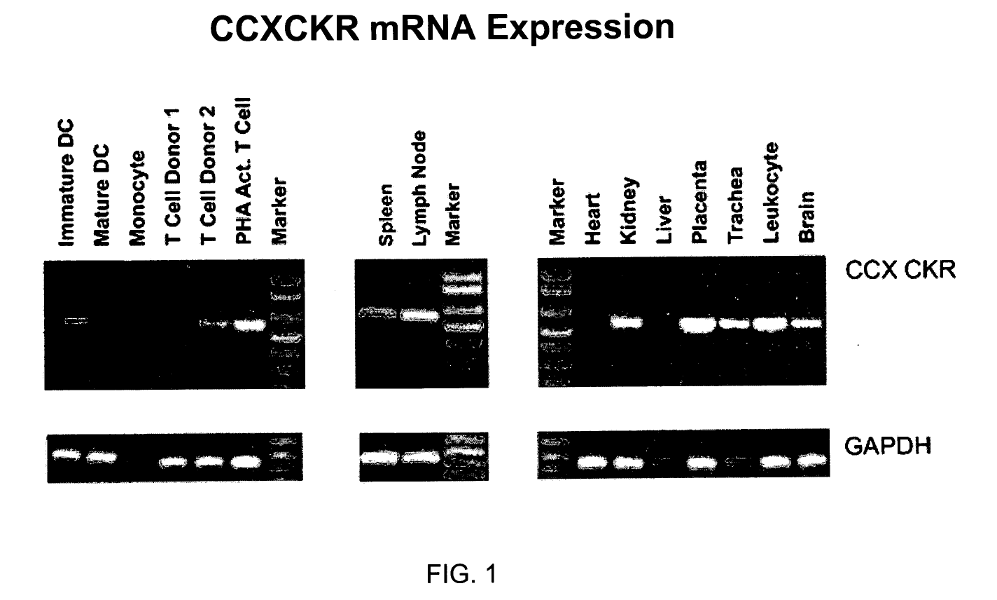

The expression of CCX CKR mRNA was determined by PCR analysis of human cDNAs as well as by RT-PCR of RNAs isolated from various tissues. First, CCX CKR expression in hematopoietic cells and tissues was investigated. Receptor expression was apparent in immature dendritic cells (DC) (derived from monocytes after treatment with GM-CSF and IL-4), primary T cells from 2 of 3 donors, and in spleen and lymph node tissue (FIG. 1). Additionally, expression was detected in non-lymphoid tissues such as heart, kidney, placenta, trachea, and brain; unfractionated leukocytes on the same panel were also positive (FIG. 1). Control PCR products for GAPDH confirmed the integrity of all starting RNA.

The observed pattern of CCX CKR overlaps with, and expands, the distribution reported for human expressed sequence tags found in the NCBI databases: These ESTs have been have been isolated from kidney, fetal heart, olfactory epithelium, and tonsillar B cells. Thus, CCX CKR seems expressed in motile cells in the periphery, as well as in lymphoid and non-lymphoid tissues. Expression of CCX CKR in a variety of cells other than those involved in cell migration indicates that the receptor plays a role in biological activities other than simply inflammatory diseases and cellular motility.

### EXAMPLE 2

### Signaling Experiments

A series of experiments were conducted to determine whether CCX CKR exhibited activities typically associated with chemokine receptors. Assays that were conducted included ligand-dependent calcium signaling, ligand-dependent phosphatidyl inositol turnover, ligand-dependent metabolic rate and ligand-dependent chemotaxis. The results from these assays were negative or inconclusive, indicating at most a low level activity.

These results indicate that a primary activity of CCX CKR is an activity other than those typically associated with chemokine receptors (e.g., cell migration).

### EXAMPLE 3

### Effect of CCX CKR Expression on Protein Expression

Studies were also conducted to assess what effect CCX CKR activity had on protein expression. This was analyzed by determining protein levels for both unstransfected HEK293 cells, and HEK293 cells transfected with CCX CKR, grown both in the presence and absence of the ligands ELC, SLC or TECK. Protein levels were determined using the BD PowerBlot™ technology developed by Becton Dickinson, and available from BD Biosciences Pharmingen. In general, this analysis involves separating proteins in cellular lysates by polyacrylamide electrophoresis and then conducting a Western blot with over 1,000 monoclonal antibodies specific for certain proteins. Antibodies that bind to a protein band on the gel are visualized using a digital camera that detects chemiluminescence from the bound antibody. The amount of protein can be determined based upon the strength of the signal. Further details regarding the application of this technology are discussed by Lorenz, P., et al. Proteomics (2003) 3:991-1002; Malakhov, M. P., et al. (2003) J. Biol. Chem. .278:16608-13; and Yoo, G. H., et al. (2002) Clin Cancer Res. 8:3910-21.

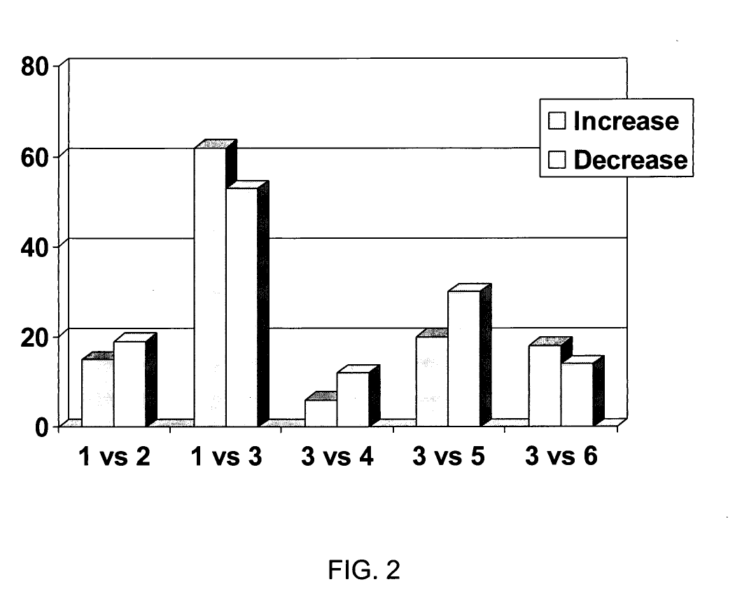

FIG. 2 provides a chart that provides a comparison of the number of proteins that were increased and decreased under different sets of conditions. Working from left to right, the first grouping shows that contacting untransfected HEK293 cells with ELC, a CCX CKR ligand, has a relatively minor effect on protein expression levels. The second grouping demonstrates that simply transfecting HEK293 cells with CCX CKR resulted in a significant change in protein expression, which the expression level of a significant number of genes both increasing and decreasing. The last three groupings indicate that the addition of any of three CCX CKR ligands (ELC, SLC or TECK) to CCX CKR-transfected cells resulted in only a modest change in protein expression relative to cells simply transformed with CCX CKR.

These results are the reverse of what is typically observed for chemokine receptors. Whereas CCX CKR itself, in the absence of ligands, was able to cause a significant change in protein expression, most chemokine receptors can cause such changes only once activated by ligand binding. For most chemokine receptors, transfection itself has only a limited effect on gene expression. These results thus suggest that unlike most chemokine receptors that CCX CKR is autoactive and can promote high levels of protein turnover in cells, even in the absence of ligand.

### EXAMPLE 4

### Effect of CCX CKR Expression on Cell Growth

Another set of experiments were conducted to ascertain whether CCX CKR plays a role in cell growth. One experiment involved culturing (1) untransfected HEK293 cells (ATCC #CRL 1573) (control), (2) HEK 293 cells transfected with CCX CKR as a polyclonal mixture, and (3) three different clones of HEK293 stably transfected with CCX CKR, thus giving a total of 6 different cell cultures. Each of these cultures were obtained by seeding 50,000 cells/well in triplicate. The three different clones (clones 9, 12 and 13) were obtained by a limited dilution of the polyclonal mixture of cells.

Cells were allowed to attach for 12 hours in growth medium (Dulbecco's minimal essential medium with 10% fetal bovine serum), then cultured in the absence of fetal bovine serum for 12 hours to synchronize the cell cycle. Growth medium was then added back to cells, and plates returned to the incubator. Each day for 6 days, cells were removed with trypsin and counted with a hemacytometer.

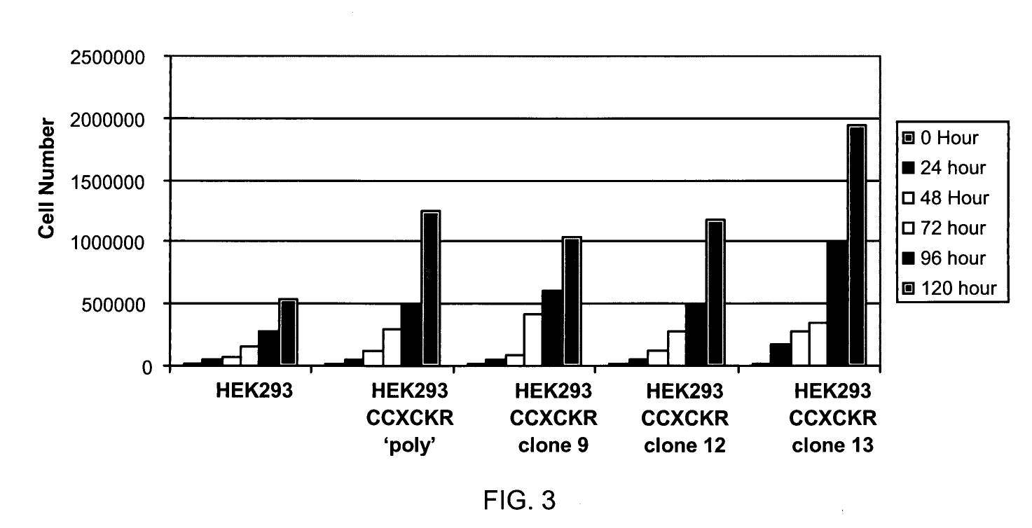

Results from this experiment are shown in FIG. 3, which is a plot of cell number each day for each cell type. This plot thus represents a growth curve for each of the six cell cultures. The chart shows that simply transfecting cells with CCX CKR to increase CCX CKR expression was sufficient to significantly increase cell growth. The increase in cell growth was observed with both the polyclonal transformation and each of the three individual clones obtained from the polyclonal transformation mixture. Because these experiments were conducted without added CCX CKR ligand, these results also indicate that CCX CKR is autoactive, i.e., able to promote cellular growth even in the absence of ligand.

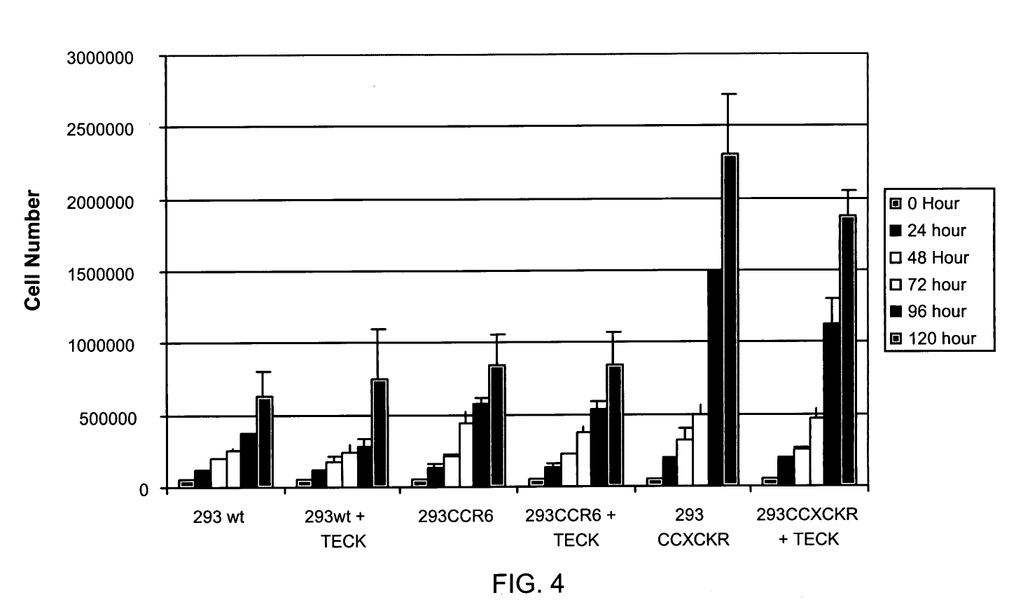

Another set of experiments were conducted using the same procedure just described to address two issues: (1) whether the autoactive response observed in the first set of experiments was simply a consequence of the transformation process, and (2) to determine what effect ligands had on the ability of CCX CKR to promote cellular growth. In this experiment, cellular growth curves were determined for untransfected HEK293 cells, HEK293 cells transfected with the chemokine receptor CCR6 and cells transfected with CCX CKR. Growth curves were determined in the presence and absence of the CCX CKR ligand TECK, which was included or omitted from the growth medium that was added back to the cells once cell cycles had been synchronized (see FIG. 4).

The chart shown in FIG. 4 illustrates several points. First, consistent with the results from the first set of experiments, simply transfecting HEK293 cells with CCX CKR was sufficient to induce significant cellular growth. These results thus support the conclusion that CCX CKR is autoactive. Second, the addition of TECK with cells expressing CCX CKR had a modest effect on cell growth activity, with perhaps even a slight decrease in cell growth. This again is consistent with CCX CKR being an autoactive receptor, with ligands apparently acting to fine tune the level of activity. Third, the results show that the growth activity observed with CCX CKR is not simply due to the transformation process or presence of plasmid in the cells because cells transformed with CCR6 (transformed with the same plasmid as the CCX CKR transformed cells) did not show a significant increase in cellular growth.

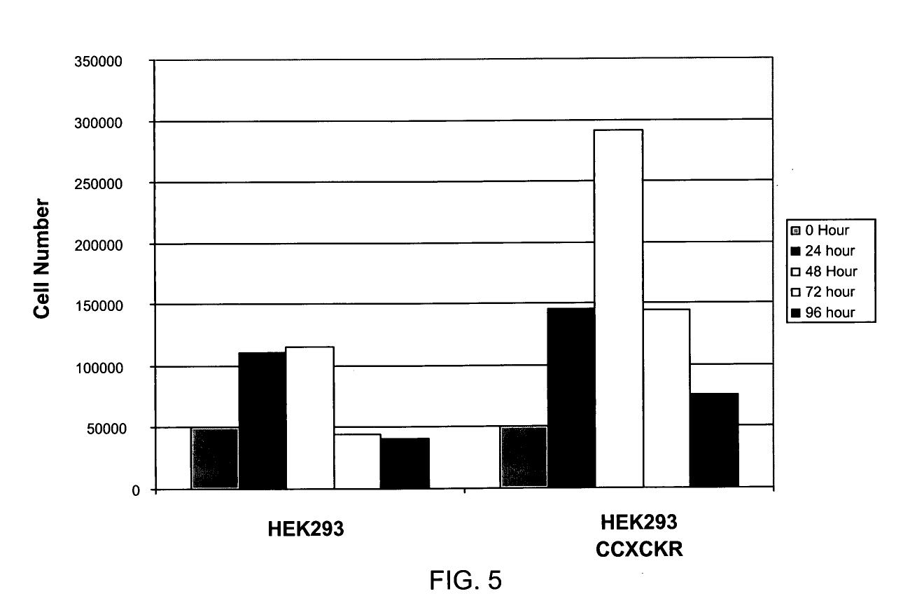

Another set of experiments were performed to confirm that the results indicating that CCX CKR is autoactive were not simply due to the presence of a CCX CKR ligand in the serum contained in the growth medium. In these experiments, untransformed HEK293 cells and HEK293 cells transformed with CCX CKR were grown in the absence of serum. As shown in FIG. 5, the untransformed cells grew initially, but then began to die due to lack of nutrients. This same general trend was observed with the transformed cells, but the initial growth rate was significantly increased, consistent with the other results indicating that CCX CKR is autoactive and capable of inducing cellular growth. This experiment thus confirmed that the correlation between CCX CKR expression and growth activity could not simply be attributed to the presence of ligands in the serum.

So collectively the results from this series of experiments show that CCX CKR is an autoactive receptor, whose expression alone is sufficient to promote cell growth significantly. Consequently, cell growth can be promoted by increasing the activity or expression level of the receptor. Binding of ligands does not appear to be required for triggering the promotion of cell growth, but may fine tune the level of activity.

All references cited herein are incorporated herein by reference in their entirety and for all purposes to the same extent as if each individual publication or patent application was specifically and individually indicated to be incorporated by reference in its entirety for all purposes.

1. A method for screening for a modulator of cell proliferation, comprising:
(a) contacting a population of cells that comprise a recombinant construct encoding CCX chemokine receptor (CCX CKR) with a test agent; and
(b) determining the effect of the test agent on proliferation of the cell population.

2. The method of claim 1, wherein contacting is done in the absence of a ligand that modulates CCX CKR activity.

3. The method of claim 2, wherein determining comprises counting the number of cells in the cell population using a hemacytometer at a plurality of time points.

4. The method of claim 2, wherein determining comprises conducting an assay that measures the metabolic activity of the population of cells.

5. The method of claim 2, wherein determining comprises conducting an assay that measures DNA replication in the population of cells.

6. The method of claim 2, wherein determining comprises conducting an assay that measures the concentration of a cell cycle antigen in the population of cells, wherein the antigen is specific to proliferating cells.

7. The method of claim 2, wherein determining comprises comparing a measure of the proliferation of the cell population with a measure of proliferation of a control population of cells.

8. The method of claim 1, wherein the agent is an antibody.

9. A method for screening for a modulator of an activity of CCX chemokine receptor (CCX CKR), comprising:
(a) contacting a cell comprising a recombinant construct encoding CCX CKR with a test agent in the absence of a ligand that specifically binds CCX CKR; and
(b) determining the effect of the test agent on the CCX CKR activity.

10. A method for stimulating cell growth comprising activating the expression and/or activity of CCX CKR in a cell.

11. The method of claim 10, wherein activation comprises introducing a nucleic acid construct encoding CCX CKR into the cell, whereby expression of CCX CKR in the cell is increased.

12. The method of claim 10, wherein activating is done in the absence of a ligand that specifically binds CCX CKR.

13. The method of claim 10, wherein the method is conducted in vitro.

14. The method of claim 10, wherein the method is conducted ex vivo.

15. The method of claim 10, wherein activation comprises activating expression and/or activity of CCX CKR in a population of cells, whereby a tissue is formed.

16. The method of claim 15, wherein the population of cells are bone marrow cells.

17. The method of claim 15, wherein the population of cells are endothelial cells.

18. The method of claim 15, wherein the population of cells are heart cells or cardiomyocytes.

19. The method of claim 15, further comprising transplanting the tissue to a patient.

20. A method for treating a disease associated with cell proliferation, comprising administering to a patient having or susceptible to the disease an agent that inhibits the cell proliferative activity of CCX chemokine receptor (CCX CKR) or its expression.

21. The method of claim 20, wherein the disease is leukemia.

22. The method of claim 20, wherein the disease is kidney or liver cancer.

23. The method of claim 20, wherein the disease is a brain tumor.
